(19) United States
(12) Patent Application Publication
(10) Pub. No.: US 2019/0000353 A1
(43) Pub. Date: Jan. 3, 2019

(54) VAGINAL CANAL MEASUREMENT DEVICE

(71) Applicant: James Carty, Santa Monica, CA (US)

(72) Inventor: James Carty, Santa Monica, CA (US)

(21) Appl. No.: 15/636,831

(22) Filed: Jun. 29, 2017

(51) Int. Cl. A61B 5/107; A61B 17/42; A61B 5/00

(52) U.S. Cl. CPC A61B 5/1076; A61B 5/036; A61B 5/4337; A61B 17/42

(57) ABSTRACT

The Vaginal Canal Measurement Device is the only product of its kind to give an accurate evaluation of women's vaginal opening without the need for doctor to patient contact or guessing. The Vaginal Canal Measurement Device is uniquely designed to be a single-use product, to minimize exposure to bacteria.

### BACKGROUND OF THE INVENTION

### Field of the Invention

The present invention is a medical device. More specifically, the present invention is a vaginal canal measurement device for single as well as multiple uses.

### Description of the Related Art

There are currently no feasible products on the market that facilitates measuring the diameter size of the vaginal canal. Measurement of a woman's pelvic cavity is needed during the process of labor in order to ensure a dilation of ten centimeters, as recommended, to reduce the risk of injuries that can occur to both the mother and baby. Currently, doctors use the digital palpation method, which consists of inserting a gloved hand into the vaginal canal and using the index and middle finger to estimate the measurement. However, this technique causes concerns due to its uncertainty in measurements and its potential for health hazards.

### SUMMARY OF THE INVENTION

The present invention is a vaginal canal measurement device for single as well as multiple uses.

The vaginal canal measurement device includes a prong vaginal insert and a millimeter scale.

It is an object of the present invention to provide a vaginal canal measurement device that can measure cervical expansion accurately and used to measure the canal for vaginal tightening.

It is an object of the present invention to provide a vaginal canal measurement device that comprises a disposable probe with a millimeter measuring scale.

It is an object of the present invention to provide a vaginal canal measurement device that is designed to be inserted into the vaginal canal closed and once inside the tool independently and gently opens causing the scale to slide and give an exact measurement reading.

It is an object of the present invention to provide a vaginal canal measurement device that is designed to measure the width and depth of the vaginal canal.

It is an object of the present invention to provide a one-time use sterile item that reduces chances of infections caused by penetrations of the canal.

It is an object of the present invention to provide a vaginal canal measurement device that measures a woman's pelvic cavity during the process of labor in order to ensure a dilation of ten centimeters, as recommended, to reduce the risk of injuries that can occur to both the mother and baby.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a front view of a vaginal canal measurement device in a closed vaginal canal, in accordance with one embodiment of the present invention.

FIG. 2 illustrates a front view of a vaginal canal measurement device in an open vaginal canal, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

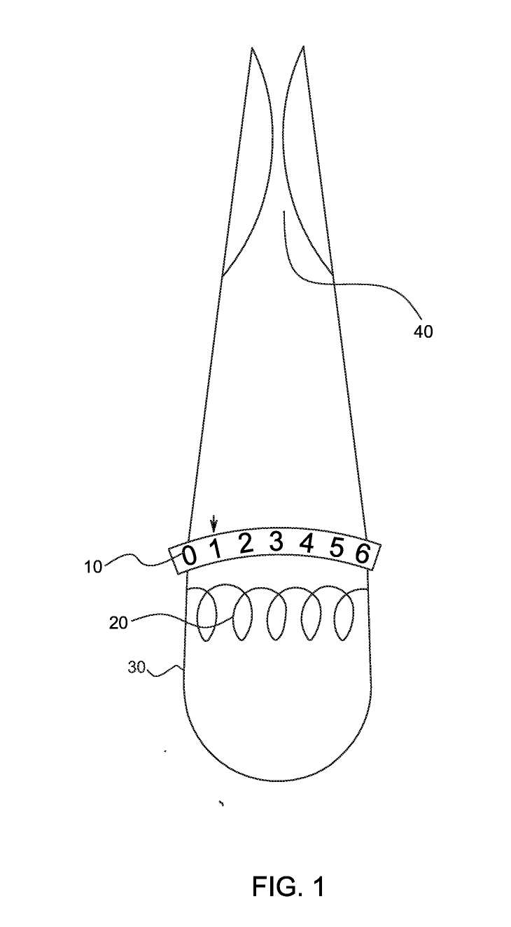

Referring now to FIG. 1, illustrated is a front view of a vaginal canal measurement device in a closed vaginal canal, in accordance with one embodiment of the present invention.

In FIG. 1, illustrated is a front view of a vaginal canal measurement device for single as well as multiple uses in a closed vaginal canal 40, including a millimeter scale 10, a spring 20 and a prong body 30. 1. A vaginal canal measurement device for single as well as multiple uses with a prong vaginal insert a scale in a selected one of millimeter, centimeters and inches. The scale connected to the prong vaginal insert

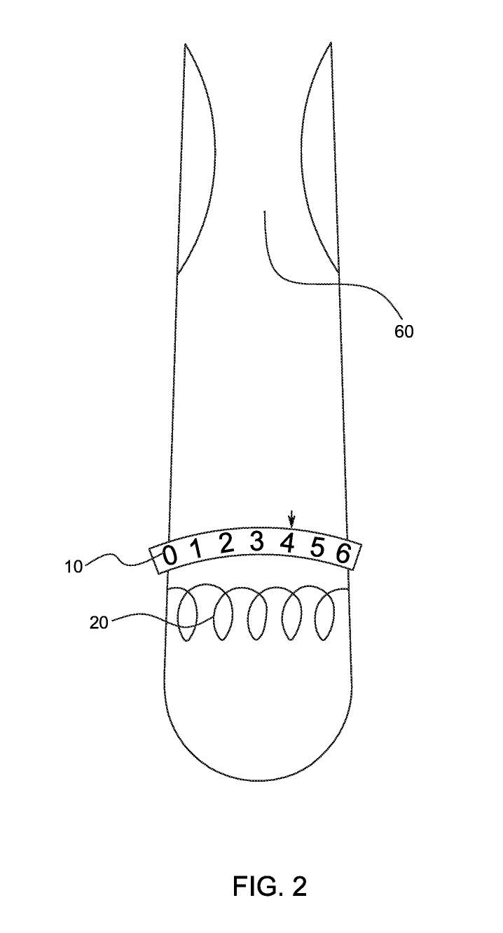

Referring now to FIG. 2, illustrated is a front view of a vaginal canal measurement device in an open vaginal canal, in accordance with one embodiment of the present invention.

In FIG. 2, illustrated is a front view of a vaginal canal measurement device in an open vaginal canal 60, including a millimeter scale 10, a spring 20 and a prong body 30. Measuring cervical expansion accurately and to measure the canal for vaginal tightening is another use of the device. With a disposable probe the present invention is designed to be inserted into the vaginal canal closed and once inside the tool independently and gently opens causing the scale to slide and give an exact measurement reading.

5. The vaginal canal measurement device according to claim 1, wherein said device is designed to measure the width and depth of the vaginal canal.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A vaginal canal measurement device for single as well as multiple uses, said device comprising:
a prong vaginal insert;
a scale in a selected one of millimeter, centimeters and inches said scale connected to said prong vaginal insert.

2. The vaginal canal measurement device according to claim 1, wherein said device measures cervical expansion accurately and to measure the canal for vaginal tightening.

3. The vaginal canal measurement device according to claim 1, wherein said device comprises a disposable probe.

4. The vaginal canal measurement device according to claim 2, wherein said device is designed to be inserted into the vaginal canal closed and once inside the tool independently and gently opens causing the scale to slide and give an exact measurement reading.

5. The vaginal canal measurement device according to claim 1, wherein said device is designed to measure the width and depth of the vaginal canal.

6. The vaginal canal measurement device according to claim 1, wherein said device is a one-time use sterile item that reduces chances of infections caused by penetrations of the canal.

7. The vaginal canal measurement device according to claim 1, wherein said device measures a woman's pelvic cavity during the process of labor in order to ensure a dilation of ten centimeters, as recommended, to reduce the risk of injuries that can occur to both the mother and baby.
